(19) United States
(12) Patent Application Publication
(10) Pub. No.: US 2013/0296412 A1
(43) Pub. Date: Nov. 7, 2013

(54) Artemisinin-Like Derivatives with Cytotoxic and Anti-Angiogenic Properties

(71) Applicant: Frans Herwig Jansen, Oud-Turnhout (BE)

(72) Inventor: Frans Herwig Jansen, Oud-Turnhout (BE)

(73) Assignee: DAFRA PHARMA N.V., Turnhout (unknown)

(21) Appl. No.: 13/813,741

(22) PCT Filed: Aug. 3, 2011
(86) PCT No.: PCT/EP11/63363

§ 371 Date: Apr. 8, 2013

(30) Foreign Application Priority Data
Aug. 3, 2010 (EP) PCT/EP2010/061264

(51) Int. Cl. C07D 493/18

(52) U.S. Cl. CPC C07D 493/18
USPC 514/450; 549/348

(57) ABSTRACT

The present invention relates to novel artemisinin-like derivates, and especially dihydroartemisinin derivates and pharmaceutical compositions comprising the present compounds. The present invention further relates to the use of the present compounds for the treatment of cancer, especially by oral administration. Especially, the present invention relates to dihydroartemisinin compounds (DHA) substituted by, through an ester linkage by a linear or branched C₁ to C₆ alkyl optionally substituted by one or more halogens. Especially preferred substituents are acetate, propionate, isopropionate, butyrate and isobutyrate.

The present invention relates to novel artemisinin-like derivates, and especially dihydroartemisinin derivates and pharmaceutical compositions comprising the present compounds. The present invention further relates to the use of the present compounds for the treatment of cancer, especially by oral administration.

Artemisinin is a natural product of the plant Artemisia annua L. Reduction of artemisinin yields the more active dihydroartemisinin (DHA), a compound which is thermally less stable. DHA can be further converted into different derivatives, including, for example, artesunate and artemether, which are generally referred to as artemisinins.

Artemisinins are widely known for their potent anti-malarial activity, but also have efficacy in the treatment of several protozoal and schistosomal infections. Artemisinin-like compounds exhibit a wide spectrum of biological activities, including, for example, anti-angiogenic, anti-tumorigenic, and even anti-viral, all of which are of medical importance.

The anti-tumorigenic activity of the drug is believed to be partly due to iron-dependent generation of reactive oxygen species, as well as alkylation of proteins and DNA. The underlying molecular mechanism by which artemisinins suppress angiogenesis, which in turn, contributes to the anti-tumor activities, are less clear. Nonetheless, direct effects on angiogenesis and lymphangiogenesis have been described.

Artemisinins inhibit endothelial cell proliferation, cell migration and endothelial tube formation, at least partly by inducing apoptosis. They also interfere with synthesis of vascular endothelial growth factors, possibly via suppression of hypoxia inducible factor (HIF) activation.

Despite of the therapeutic utility of artemisinins in treating malaria, resistant strains of the malaria parasites are emerging, mostly in western Cambodia where treatment failure rates after combination therapy have exceeded 10%. The mechanisms of resistance are largely unknown, but may replicate some of those that become active in cancer cells as they develop chemo-resistance. These include, among others, mutations in target proteins, resistance to apoptosis, and increased drug efflux via transporters. The latter mechanism is known to be used by parasites to enhance the clearance of drugs, and the multidrug resistance-conferring ATP-binding cassette (ABC) transporter, P-glycoprotein (P-gp) has been implicated. Increased expression of ABC transporters such as P-gp may also enable tumor endothelial cells to escape from anti-angiogenic treatment.

It is an object of the present invention, amongst other objects, to provide novel artemisinin-like compounds, and especially dihydroartemisinin derivates, with improved clinical efficacy and/or other pharmaceutical properties as compared to non-substituted artemisinin, dihydroartemisinin and artesunate. The clinical efficiency of the present compounds is, for example, in the field of cancer treatment, especially by inhibiting angiogenesis.

The above object, amongst other objects, is met by the present invention through the compounds and formulations described in the appended claims.

Especially, the above object, amongst other objects, is met by the present invention through compounds according to the general formula:

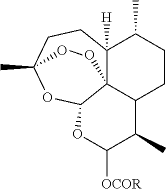

wherein R is a linear or branched C₁ to C₆ alkyl optionally substituted by one or more halogens.

The present inventors surprisingly discovered that the above compounds are easily synthesized, stable at room temperature, overcome drug-resistance pathways, and/or are more active in vitro and in vivo than the commonly used artesunate. The provision of the present compounds enables safer and more effective strategies to treat a range of infections and cancer.

According to a preferred embodiment, the present compounds comprise an R group, either linear or branched, chosen from the group consisting of methyl (C₁), ethyl (C₂), propyl (C₃), and butyl (C₄).

Examples of preferred linear alkyl groups are methyl (CH₃), ethyl (C₂H₅) or propyl (C₃H₇). The corresponding substituents at the hydroxyl group (—OH) of DHA are in this case generally designated as ethanoate (CH₃), propanoate or propionate (C₂H₅), and butyrate (C₃H₇), respectively.

Examples of preferred branched alkyl groups are isopropyl (CH(CH₃)₂) and isobutyl (C(CH₃)₃). The corresponding substituents at the hydroxyl group (—OH) of DHA are in this case generally designated as isopropanoate or isopropionate (CH(CH₃)₂) and isobutyrate (C(CH₃)₃), respectively.

A preferred halogen substituent of the present R groups is Cl.

According to the present invention, especially preferred R groups are moieties chosen from the group consisting of CH₃, CHCl₂, C₂H₅, C₃H₇, and CH(CH₃)₂.

The compounds according to the present invention are especially suitable to be used in oral formulations allowing oral administration. According to another aspect, the present invention relates to oral formulations comprising a present compound and a filler or one ore more fillers. A suitable fillers according to the present invention is a filler mixture sold under the trade name Prosolv®SMCC90 (JRS Pharma, Germany).

According to a preferred embodiment of this aspect of the present invention, the oral formulation comprises 50% to 90% (w/w), such as 55%, 60%, 65%, 70%, 75%, 80% or 85%, of a present compound.

Preferably, the present oral formulation is in the form of a tablet or capsule. The tablets or capsules according to the present invention preferably comprise 80 mg to 220 mg DHA-propionate, such as 85 mg, 90 mg, 95 mg, 100 mg, 105 mg, 110 mg, 115 mg, 120 mg, 130 mg, 140 mg, 150 mg, 160 mg, 170 mg, 180 mg, 190 mg, 200 mg, or 210 mg.

According to yet another aspect, the present inventions relates to the use of the present compounds or oral formulations for the treatment of cancer. The present treatment of cancer preferably comprises treatment of cancer by inhibiting angiogenesis.

According to an especially preferred embodiment of this aspect, the present invention relates to the treatment of cancer by oral administration.

Below, the present invention will be further detailed in the examples of preferred embodiments of the present invention. In the examples, reference is made to figures wherein:

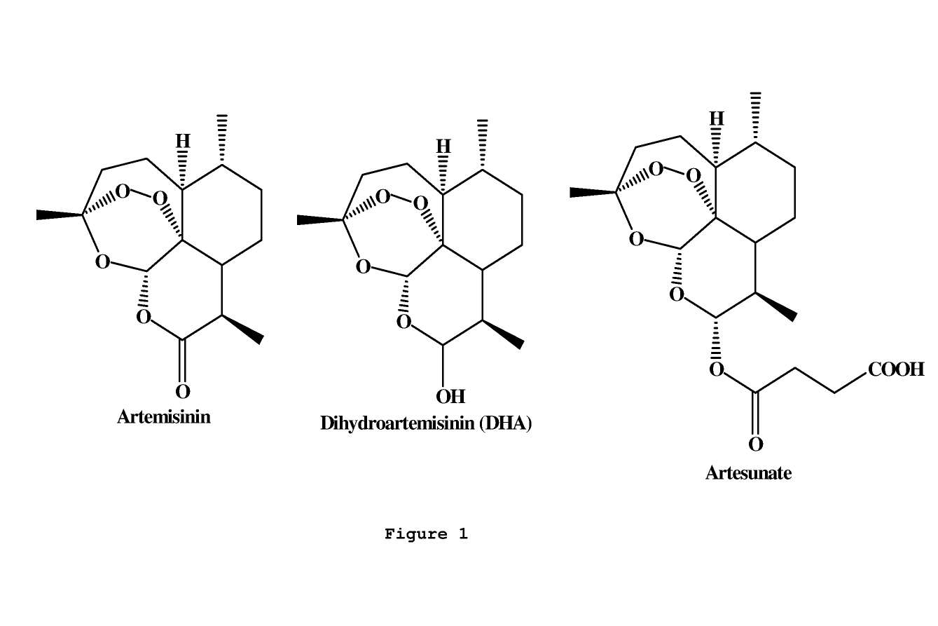

FIG. 1: shows the structures of artemisinin, DHA and artesunate.

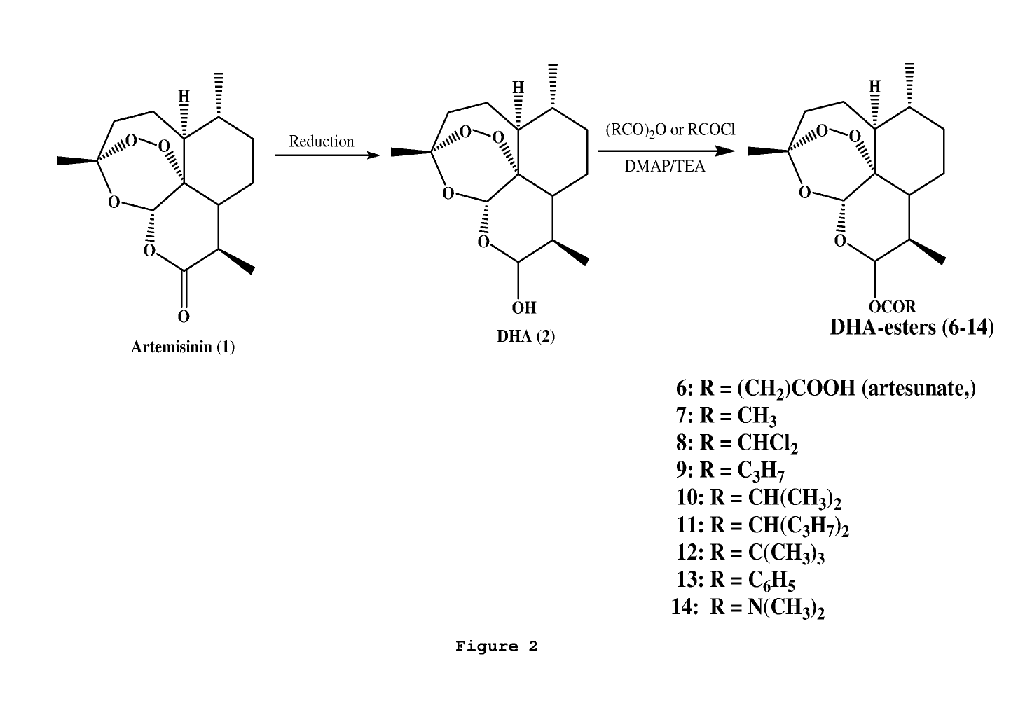

FIG. 2: shows the conversion of DHA into esters.

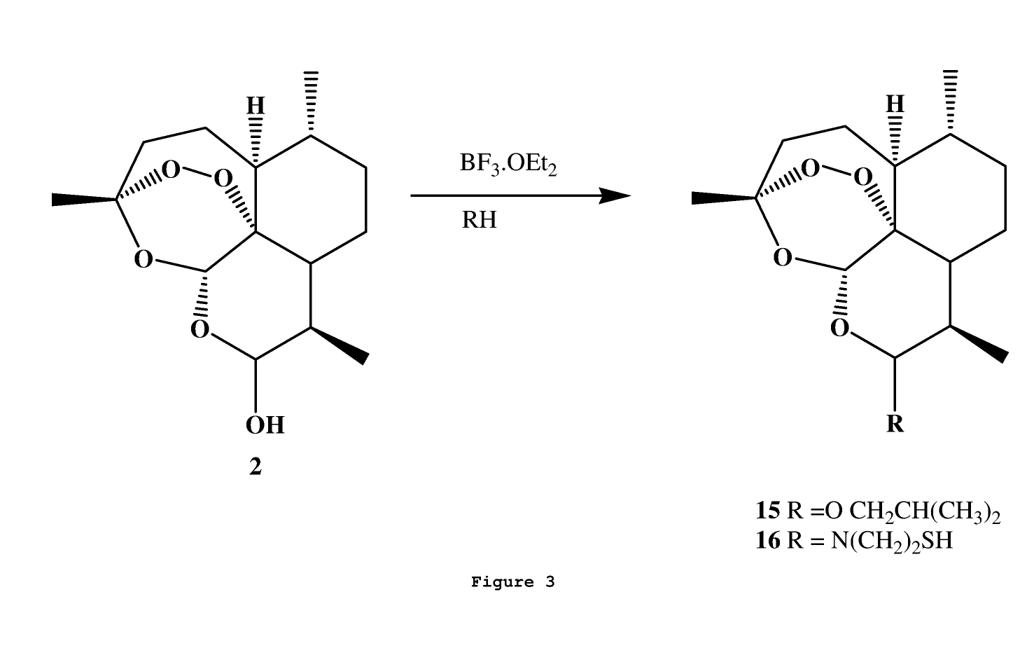

FIG. 3: shows the conversion of DHA into ether and amine.

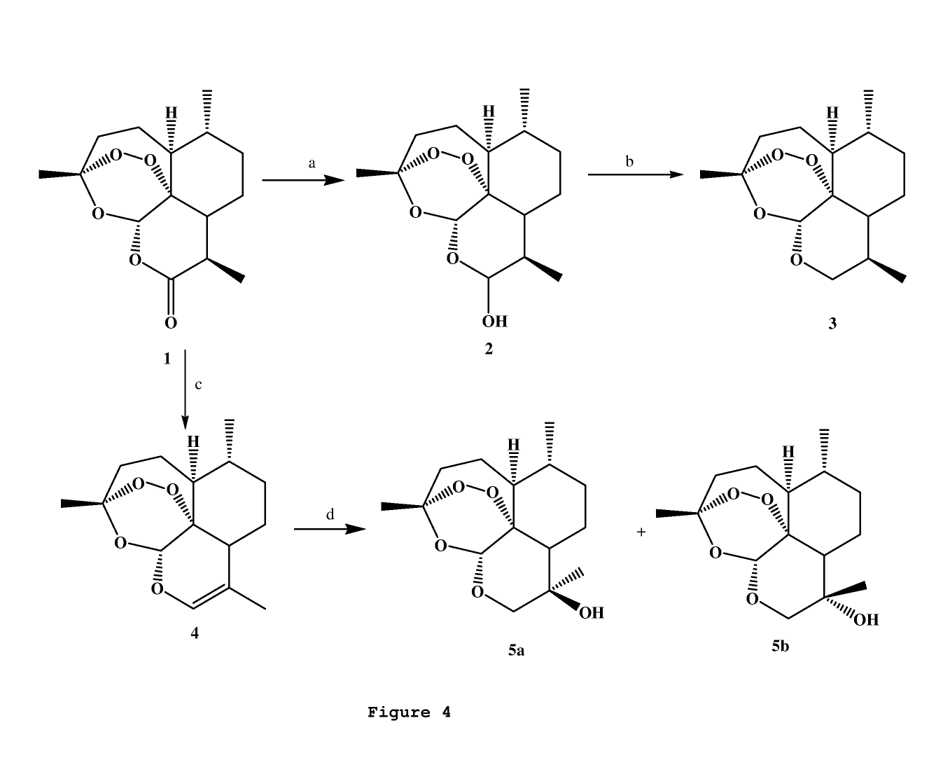

FIG. 4: shows a synthesis scheme for compounds 3, 4, and 5. Reagents and conditions: (a) NaBH₄, THF; (b) BF₃.OEt₂/Et₃SiH, CH₂Cl₂; (c) BF₃.OEt₂, CH₂Cl₂; (d) i. BH₃ᵣ THF; ii. 3M NaOHaq, H₂O₂ 30%, THF.

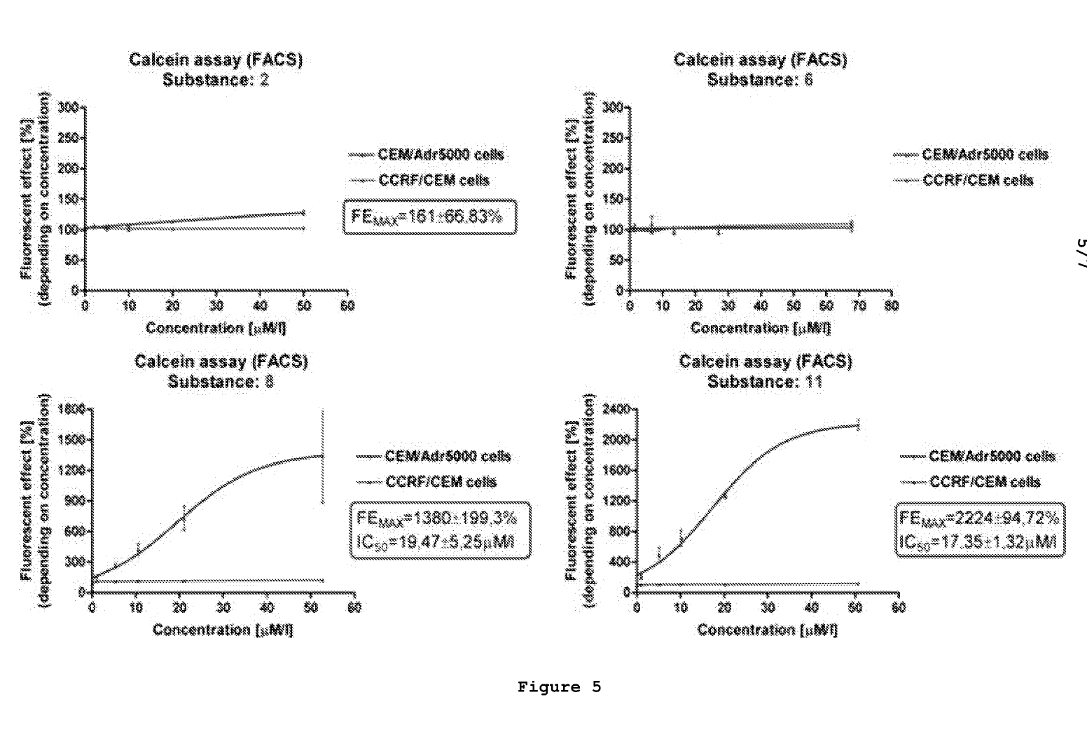

FIG. 5: shows inhibition of calcein ametoxymethylester efflux from human leukemia CCRF/CEM and CEM/Adr5000 cells by different concentrations of the testing substances—derivatives of artesunate. The intracellular accumulation of calcein inside the cells is measured by using FACS analysis. The points indicate mean values of fluorescent effect, vertical lines show standard error calculated on the base of two independent experiment replicates. The effect corresponds to a control of cells which were treated only with calcein.

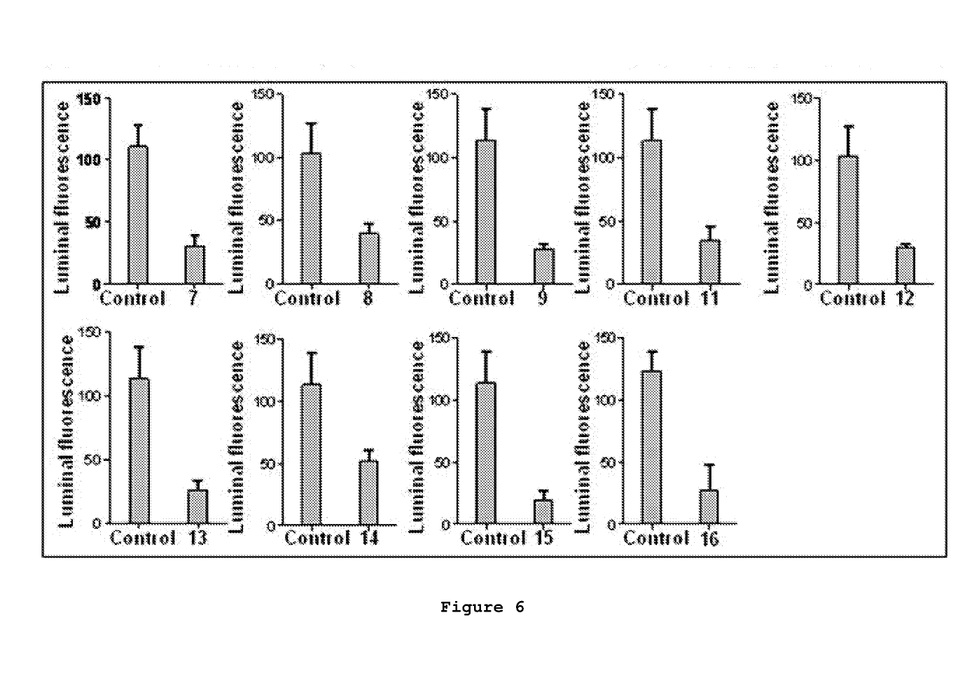

FIG. 6: shows transport of the P-gp substrate NBD-CSA into porcine brain capillary lumens in the absence of control and presence of testing substances.

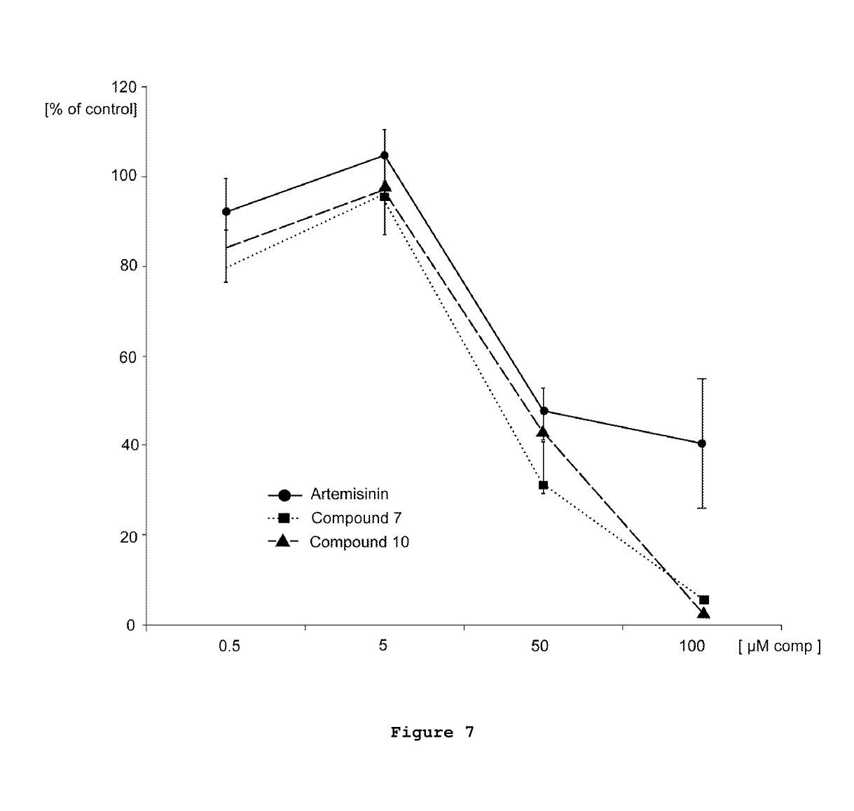

FIG. 7: shows Optical density (OD) as a measure of viable cells at various concentrations of compounds 7, 10 and artemisinin, expressed as percentage of control (VEGF) treated HUVECs. Increasing levels of artemisinin-like compounds strongly inhibit proliferation/survival of HUVECs even in the presence of VEGF. Error bars=SEM.

### EXAMPLE 1

### Introduction

This example describes the synthesis of several novel artemisinin-like compounds, their in vitro cytotoxic effects, their capacity to alter P-gp function, and their in vivo anti-angiogenic properties. All artemisinin-like compounds synthesized and tested were based on dihydroartemisinin (DHA), a breakdown product of artesunate. The biochemical approach was feasible, because the lactol of DHA can be converted into different derivatives, such as ethers and esters, allowing synthesis of a range of different DHA derivatives. The structures of artemisinin, dihydroartemisinin (DHA) and artesunate are shown in FIG. 1.

### Material and Methods

### Chemistry

Materials and reagents were purchased from Acros Organics, Beerse, Belgium or Aldrich. Tris-(2-aminoethyl)-amine polystyrene resin was obtained from Nova biochem. Nuclear magnetic resonance (NMR) spectra were recorded on a Bruker Avance DRX-400 spectrometer (400 MHz). Coupling constants (J) are reported in Hz. Column chromatography was performed on a Flashmaster II (Jones Chromatography) with Isolute columns pre-packed with silica gel (30e90 mM) for normal phase chromatography. Melting points were determined with a capillary melting point apparatus (Buchi 510, BUCHI, Flawil, Switzerland) and are uncorrected. Electrospray Ionization (ESI) mass spectra were acquired on an ion trap mass spectrometer (Bruker Daltonics esquire 3000 plus). LC-MS spectra were recorded on an Agilent 1100 Series HPLC system equipped with a HILIC Silica column (2.1 100 mm, 5 mm, Atlantis HILIC, Waters) coupled with a Bruker Daltonics esquire 3000 plus mass spectrometer (solvent A: H₂O with 0.1% formic acid, solvent B: ACN with 0.1% formic acid, gradient 2: 90% B to 40% B, 12 min., 0.2 ml/min). Analytical TLC was performed on pre-coated silica gel plates (60 F254, 0.2 mm thick, VWR), visualization of the plates was accomplished using UV light and/or Iodine staining.

The dried solvents were purchased from Acros Organics. Artemisinin, dihydroartemisinin and artesunate were provided by Dafra Pharma R&D (Turnhout, Belgium). Anhydrodihydroartemisinin (4), Deoxoartemisinin (3), 10-Dihydroartemisinyl acetate (7), Compound 5a synthesized by a modified procedure (NaOH/H₂O₂ were used as oxidizing agents), 10-dihydroartemisinyl benzoate (13) with small modification (instead of benzoylchloride, the benzoic anhydride was used with catalytic amount of DMAP) were prepared as previously described.

### Synthesis of 10-Dihydroartemisinyl 2′,2′-Dichloroacetate (8)

DMAP (0.6 g, 4.9 mmol) and dichloroacetic anhydride (6.0 g, 25 mmol) were added to a stirred solution of DHA (5 g, 17.6 mmol) in dichloromethane (300 ml) at 0° C. and the reaction mixture was slowly brought to room temperature and stirred for 6 hours, during which time, all DHA was consumed. The solvent was removed under reduced pressure and the residue was purified by flash chromatography with ethyl acetate/hexane (10:90 to 50:50) to provide the product dense liquid (3.82 g, 55%).

¹HNMR (400, CDCl₃) d 0.86 (d, J=7.0 Hz, 3H, 9-Me), 0.97 (d, J=5.95 Hz, 20 3 H, 6-Me), 1.45 (s, 3H, 3-Me), 1.23-1.94 (m, 9H), 2.04 (ddd, J=14.5, 5.0, 3.0 Hz, 1H), 2.39 (ddd, J=14.5, 5.0, 3.0 Hz, 1H), 2.55 (m, 1H, H-9), 5.40 (s, 1H, H-12), 5.90 (d, J=10.0 Hz, 1H, H-10), 6.25 (s, 1H, COCHCl₂); SIMS (m/z) 396.3 (M+H)⁺.

### Synthesis of 10-Dihydroartemisinyl Butyrate (9)

DMAP (0.6 g, 4.9 mmol) and butyric anhydride (4.0 g, 25 mmol) were added to a stirred solution of DHA (5 g, 17.6 mmol) in dichloromethane (300 ml) at 0° C. and the reaction mixture was slowly brought to room temperature and stirred for 8 hours, during which time, all DHA was consumed. The solvent was removed under reduced pressure and the residue was purified by flash chromatography with ethyl acetate/hexane (10:90 to 50:50). Re-crystallization from ethyl acetate/hexane provided white big crystals (5.9 g, 95%), m.p. 81-85° C.

¹HNMR (400, CDCl₃) d 0.86 (d, J=7.0 Hz, 3H, 9-Me), 0.97 (d, J=5.95 Hz, 3H, 6-Me), 1.17-1.24 (m, 6H), 1.45 (s, 3H, 3-Me), 1.23-1.94 (m, 9H), 2.04 (ddd, J=14.5, 5.0, 3.0 Hz, 1H), 2.39 (ddd, J=14.5, 5.0, 15 3.0 Hz, 1H), 2.55 (m, 1H, H-9), 2.68 (m, 1H, COCH), 5.45 (s, 1H, H-12), 5.850 (d, J=10.0 Hz, 1H, H-10); EIMS (m/z) 355.4 (M+H)⁺.

### Synthesis of 10-Dihydroartemisinyl propionate (9a)

Synthesis of 10-Dihydroartemisinyl propionate was performed as described for 10-Dihydroartemisinyl Butyrate except propionic anhydride was used instead of butyric anhydride.

¹HNMR of 10-Dihydroartemisinyl propionate ¹HNMR (400, CDCl₃) d 0.91 (d, J=7.0 Hz, 3H, 9-Me), 1.03 (d, J=5.95 Hz, 3H, 6-Me), 1.17-1.24 (m, 6H), 1.50 (s, 3H, 3-Me), 1.23-1.94 (m, 7H), 2.04 (ddd, J=14.5, 5.0, 3.0 Hz, 1H), 2.39 (ddd, J=14.5, 5.0, 15 3.0 Hz, 1H), 2.55 (m, 1H, H-9), 2.68 (m, 1H), 5.51 (s, 1H, H-12), 5.87 (d, J=10.0 Hz, 1H, H-10); EIMS (m/z).

### Synthesis of 10-Dihydroartemisinyl Butyrate (10)

DMAP (0.6 g, 4.9 mmol) and isobutyric anhydride (4.0 g, 25 mmol) were added to a stirred solution of DHA (5 g, 17.6 mmol) in dichloromethane (200 ml) at 0° C. and the reaction mixture was slowly brought to room temperature and stirred for 8 hours, during which time, all DHA was consumed. The solvent was removed under reduced pressure and the residue was purified by flash chromatography with ethyl acetate/hexane (10:90 to 50:50) to provide the product dense liquid (5.2 g, 84%).

¹HNMR (400, CDCl₃) d 0.86 (d, J=7.0 Hz, 3H, 9-Me), 0.97 (d, J=5.95 Hz, 3H, 6-Me), 1.17-1.24 (m, 6H) 1.45 (s, 3H, 3-Me), 1.23-1.94 (m, 9H), 2.04 (ddd, J=14.5, 5.0, 3.0 Hz, 1H), 2.39 (ddd, J=14.5, 5.0, 15 3.0 Hz, 1H), 2.55 (m, 1H, H-9), 2.68 (m, 1H, COCH), 5.45 (s, 1H, H-12), 5.850 (d, J=10.0 Hz, 1H, H-10); EIMS (m/z) 355.4 (M+H)⁺.

### Synthesis of 10-Dihydroartemisinyl 2′-Propylpentanoate (11)

DMAP (0.5 g, 4.1 mmol) and triethylamine (3.03 g, 30 mmol) were added to a stirred solution of DHA (7.1 g, 25 mmol) in dichloromethane (400 ml). 2-Proplypentanlychloride (4.87 g, 30 mmol) at −30° C. was added, and the reaction mixture was continuously stirred for 2 hours and slowly brought to room temperature and stirred overnight. The solvent was removed under reduced pressure and the residue was purified by flash chromatography with ethyl acetate/hexane (10:90 to 50:50) to provide the product as a white solid. Re-crystallization from ethyl acetate/hexane resulted in a colorless liquid (8.19 g, 80%).

¹HNMR (400, CDCl₃) d 0.86 (d, J=7.0 Hz, 3H, 9-Me), 0.90 (t, 6H), 0.97 (d, J=5.95 Hz, 3H, 6-Me), 1.33 (m, 4H), 1.45 (s, 3H, 3-Me), 1.64 (m, 4H), 1.23-1.94 (m, 9H), 2.04 (ddd, J=14.5, 5.0, 3.0 Hz, 1H), 2.29 (t, 1H), 2.39 (ddd, J=14.5, 5.0, 15 3.0 Hz, 1H), 2.55 (m, 1H, H-9), 5.45 (s, 1H, H-12), 5.850 (d, J=10.0 Hz, 1H, H-10); EIMS (m/z) 411.5 (M+H)⁺.

### Synthesis of 10-Dihydroartemisinyl 2′,2′-Dimethylpropianate (12)

DMAP (0.5 g, 4.1 mmol) and trimethylacetic anhydride (5.59 g, 30 mmol) were added to a stirred solution of DHA (7.1 g, 25 mmol) in dichloromethane (400 ml) at 0° C. The reaction mixture was slowly brought to room temperature and stirred overnight, during which time all DHA was consumed. The crude material was washed with water (2×100 ml), and the solvent was removed under reduced pressure. The product was then re-crystallized from ethyl acetate/hexane, yielding white crystals (5.17 g, 75%), m.p. 101-104° C.

¹HNMR (400, CDCl₃) d 0.86 (d, J=7.0 Hz, 3H, 9-Me), 0.97 (d, J=5.95 Hz, 3H, 6-Me), 1.25 (s, 9H C(CH)₃), 1.45 (s, 3H, 3-Me), 1.23-1.94 (m, 9H), 2.04 (ddd, J=14.5, 5.0, 3.0 Hz, 1H), 2.39 (ddd, J=14.5, 5.0, 15 3.0 Hz, 1H), 2.55 (m, 1H, H-9), 5.45 (s, 1H, H-12), 5.850 (d, J=10.0 Hz, 1H, H-10); EIMS (m/z) 369.5 (M+H)⁺.

### Synthesis of 10-Dihydroartemisinyl N′,N′-Dimethylacetamide (14)

DMAP (0.5 g, 4.1 mmol) and dimethylcarbomoyl chloride (3.23 g, 30 mmol) were added to a stirred solution of DHA (7.1 g, 25 mmol) in dichloromethane (400 ml) at 0° C. The reaction mixture was slowly brought to room temperature and stirred for 8 hours, during which time all DHA was consumed. The crude material was washed with water (2×100 ml) and the solvent was removed under reduced pressure. The residue was purified by flash chromatography with ethyl acetate/hexane (10:90 to 90:10), yielding a white dense liquid (5.8 g, 65%).

¹HNMR (400, CDCl₃) d 0.86 (d, J=7.0 Hz, 3H, 9-Me), 0.97 (d, J=5.95 Hz, 3H, 6-Me), 1.45 (s, 3H, 3-Me), 1.23-1.94 (m, 9H), 2.04 (ddd, J=14.5, 5.0, 3.0 Hz, 1H), 2.39 (ddd, J=14.5, 5.0, 15 3.0 Hz, 1H), 2.55 (m, 1H, H-9), 2.92 (s, 3H, N(CH₃)₂, 2.98 (s, 3H, N(CH₃)₂, 5.45 (s, 1H, H-12), 5.68 (d, J=10.0 Hz, 1H, H-10); EIMS (m/z) 356.4 (M+H)⁺.

### Synthesis of 10-(2′-Butyloxy)Dihydroartemisinin (15)

Boron trifluoride-diethyl ether (3 ml) was added to a stirred solution of DHA (1, 2.56 g, 9.0 mmol) and ⁱbutanol (2.2 g, 30 mmol) in diethyl ether (100 ml). After 6 hours, the reaction mixture was quenched with saturated aqueous NaHCO₃ and dried with MgSO₄. Filtration and concentration of the filtrate gave a residue which on flash chromatography with ethyl acetate/hexane (5:95 to 10:90), yielded a white microcrystalline powder (2.05 g, 67%), m.p. 100-101° C.

¹HNMR (400, CDCl₃) d 0.86 (d, J=7.0 Hz, 3H, 9-Me), 0.97 (d, J=5.95 Hz, 3H, 6-Me), 1.08 (d, J=6.1 Hz, 3H), 1.20 (d, J=6.2 Hz, 3H), 1.45 (s, 3H, 3-Me), 1.23-1.94 (m, 9H), 2.04 (ddd, J=14.5, 5.0, 3.0 Hz, 1H), 2.39 (ddd, J=14.5, 5.0, 15 3.0 Hz, 1H), 2.55 (m, 1H, H-9), 4.0 (m, 1H, OCH(CH₃)₂), 4.87 (d, J=3.5 Hz, 1H, H-10), 5.44 (s, 1H, H-12); SIMS (m/z) 341.5 (M+H)⁺.

### Synthesis of 10-Dihydroartemisinyl Thioethylamine (16)

DHA (7.1 g, 25 mmol) and cysteamine (2.7 g, 35 mmol) were dissolved in 300 ml dichloromethane and boron trifluoride-diethyl ether (10 ml) was added slowly at 0° C. The reaction mixture was stirred for 3 hours at 0° C. and an additional 1 hour at room temperature. The reaction was quenched with 5% NaHCO₃ and extracted with dichloromethane. The solvent was removed under reduced pressure and the residue was purified by flash chromatography with ethyl acetate/hexane (10:90) to yield a brown wax product (4.7 g, 55%).

¹HNMR (400, CDCl₃) d 0.86 (d, J=7.0 Hz, 3H, 9-Me), 0.97 (d, J=5.95 Hz, 3H, 6-Me), 1.25, 1.45 (s, 3H, 3-Me), 1.23-1.94 (m, 9H), 2.04 (ddd, J=14.5, 5.0, 3.0 Hz, 1H), 2.39 (ddd, J=14.5, 5.0, 15 3.0 Hz, 1H), 2.55 (m, 1H, H-9), 2.9 (t, 2H), 3.1 (t, 2H), 4.56 (d, J=10.0 Hz, 1H, H-10), 5.31 (s, 1H, H-12); EIMS (m/z) 344.5 (M+H)⁺.

### XTT Cytotoxicity Assay

Multidrug-resistant, P-glycoprotein-overexpressing CEM/ADR5000 cells and their parental, drug-sensitive counterpart, CCRF-CEM cells were used. The cell lines were provided by Dr. Daniel Steinbach (University of Ulm, Ulm, Germany).

Doxorubicin resistance of CEM/ADR5000 was maintained as described. CEM/ADR5000 cells have previously been shown to selectively express MDR1 (ABCB1), but none of the other ATP-binding cassette (ABC) transporters. The cell lines were maintained in RPMI medium (Life Technologies) supplemented with 10% FCS in a humidified 7% CO₂ atmosphere at 37° C. Cells were passaged twice weekly. All experiments were done with cells in the logarithmic growth

Cytotoxicity was assessed using the 2,3-bis[2-methoxy-4-nitro-5-sulfophenyl]-2H-tetrazolium-5-carboxanilide inner salt (XTT) assay kit (Roche, Indianapolis, Ind.), which measures the metabolic activity of viable cells. Toxicity of compounds was determined with the Cell Proliferation Kit II (Roche Diagnostics, Mannheim, Germany), according to the manufacturer's instructions.

Fresh stock solutions of each compound were prepared in DMSO at a concentration of 100 mM, and a dilution series was prepared in DMEM. Cells were suspended at a final concentration of 1×10⁵ cells/ml, and 100 ml were aliquoted per well into a 96-well culture plate (Costar, Corning, USA). Marginal wells were filled with 100 μL of media to minimize evaporation. A row of wells with cells was left untreated and another row of wells with cells was treated with 1 μL DMSO, the latter serving as a solvent control. All studies were performed in duplicate, in a range of concentrations, and in two independent experiments with different batches of cells.

Quantification of cytotoxicity was achieved with an ELISA plate reader (Bio-Rad, Munchen, Germany) at 490 nm with a reference wavelength of 655 nm, and reported as a percentage of viability compared to untreated cells. The ligand binding module of Sigma plot software (version 10.0) was used for analysis.

### HUVEC Proliferation/Viability Assay

Single donor HUVEC cells were purchased from Lonza (Breda, Netherlands). Cells were seeded at 5,000 cells per well in 96-well microtiterplates in EGM-2EV medium (Invitrogen). Upon adherence the cells were gently washed twice with PBS and starved overnight in EGM-2EV medium with reduced FBS content (0.1%; starvation medium). The medium was then aspirated and replaced with starvation medium with or without 30 ng/ml recombinant human VEGF165 (R&D Systems) and with or without increasing concentrations of compound 1 (artemisinin), 7 and 10 (0.5-100 μM). Due to its precipitation from the cell culture medium, artesunate could not be used as a reference compound.

After 96 hrs, cell growth was quantified using the WST1 Rapid cell proliferation kit (Calbiochem), and was expressed in percentage of the control value (VEGF alone). Experiments were carried out in triplets.

### Isolation of Porcine Brain Capillary Endothelial Cells (PBCECs)

PBCECs were isolated from porcine brains as reported. Briefly, freshly isolated porcine brains were collected from the local slaughterhouse, cleaned of meninges, choroid plexus, and superficial blood vessels.

After removal of gray matter, the tissue was minced into cubes <2 mm³ and incubated in Medium 199, supplemented with 0.8 mM L-glutamine, penicillin/streptomycin (100 U/ml), 100 μg/ml gentamicin, and 10 mM HEPES, pH 7.4 (Biochrom, Berlin, Germany) with dispase II (0.5%) (Roche Diagnostics, Mannheim, Germany) for 2 h at 37° C. After centrifugation at 1000 g for 10 min at 4° C., the supernatant was discarded and the pellet was re-suspended in media containing 15% dextran (Sigma-Aldrich, Taufkirchen, Germany). Micro-vessels were separated by centrifugation at 5800 g for 15 min at 4° C. and incubated in 20 ml medium containing collagenase-dispase II (1 mg/ml) (Roche Diagnostics) for 1.5-2 h at 37° C.

The resulting cell suspension was filtered through a 150 μm Polymon® mesh (NeoLab Migge, Heidelberg, Germany) and centrifuged for 10 min at 130 g at 4° C. The cell pellet was re-suspended in media containing 9% horse serum (Biochrom) and separated on a discontinuous Percoll (Sigma-Aldrich) gradient consisting of Percoll® 1.03 g/ml (20 ml) and 1.07 g/ml (15 ml) by centrifugation at 1000 g for 10 min at 4° C.

Endothelial cells were enriched at the interface between the two Percoll solutions. Cells were collected, washed in media with 9% horse serum at 4° C., and stored with 10% DMSO in liquid nitrogen until use.

### Calcein-AM Assay

Freshly isolated or recently thawed PBCECs were incubated in DMEM/HAM's F12 1:1 (Biochrom) for 1 h at 37° C. at a cell density of 2.5×10⁶ cells/10 ml.

Test compounds were dissolved in DMSO as stock solutions and further dilutions were made with DMEM/HAM's F12 1:1 (Biochrom). DMSO concentration in the cell suspension did not exceed 1%, a concentration that was determined not to affect the assay. A range of concentrations of test compound in a volume of 300-600 μL cell suspension were added, followed by a 15 min incubation at 37° C. Calcein-AM (300 μL) (MoBiTec, Göttingen, Germany) in DMEM/HAM's F12 1:1 was added to a final concentration of 1 μM and incubated for 30 min at 37° C.

Suspensions were then centrifuged at 200 g for 5 min. cells were washed with 4° C. DMEM/HAM's F12 1:1, and centrifuged again at 200 g for 5 min. at 4° C. The supernatant was discarded and cells were lysed with 600 μL 1% Triton X100 for 10 min on ice. 100 μL of clarified cell lysate was added to 1 well of a 96-well microplate.

Fluorescence was detected with a Fluoroskan Ascent plate reader (Labsystems, Helsinki, Finland) (l(excitation)=485 nm and l(emission)=520 nm). All concentrations and controls were measured 10-12 times, at least three experiments were performed per test compound.

### Flow Cytometry

For the calcein-AM assay using flow cytometry, the cell density of suspensions in DMEM/Ham's F12 1:1 was 2.5×10⁷ cells/ml.

Intracellular fluorescence was measured using a fluorescence-activated cell sorting system (FACS: Calibur flow cytometer, Becton-Dickinson, Franklin Lakes, N.J., USA) with l(excitation)=488 nm and a 530/30 band-pass filter to collect emitted fluorescence. Gating on forward and side scatter in concert with propidium iodide staining allowed distinguishing live endothelial cells.

Twenty thousand cells were sorted in each run, and data were processed and analyzed with CellQuest (Franklin Lakes, N.J., USA). All fluorescence signals were corrected for background fluorescence. Calcein-AM auto-hydrolysis was measured in control samples (n=6) without cells. The increase in intracellular fluorescence induced by a test compound was compared to control fluorescence levels (100%), and results are reported as percentage of control.

### In Vivo Experiments

Tg(fli1:EGFP) zebrafish, which express enhanced green fluorescent protein (GFP) in their endothelial cells, were used as an in vivo model for angiogenesis. At 20 hours post-fertilization (hpf), zebrafish embryos (10 per well/condition) were bathed in fish media, containing a concentration range of each of the compounds or control. Compounds had been dissolved as stock solutions in DMSO, stored at room temperature, and serially diluted in fish media prior to use. The anti-angiogenic tyrosinase kinase inhibitor SU5416 (Pfizer), and a vehicle-alone control containing the maximum concentration of DMSO were used as controls in all experiments.

In the first sets of experiments, a broad range of concentrations were used to identify the maximum tolerable dose, based on toxicity to the embryos, visualized directly by light microscopy. Subsequent experiments were performed a minimum of two times. Live analyses of the embryos were performed under light and fluorescence microscopy at 28 hpf and 48 hpf to monitor viability, overall morphology, and pattern of swimming. Angiogenesis was evaluated visually by fluorescence microscopy.

The developmental growth and patterning of the dorsal aorta, posterior cardinal vein, intersomitic vessels (ISV), and vascular plexus (VP) were monitored, as was the heart rate, and blood flow.

### Results

### Synthesis of Compounds

To identify novel artemisinin-like compounds for evaluation of efficacy in different models, we synthesized several acetal and non-acetal derivatives of DHA were synthesized. Esters (FIG. 2) were made by reacting DHA with corresponding anhydrides or acid chloride in basic medium in the presence of triethylamine. The ether and amine (FIG. 3) were synthesized by reacting DHA with a Levis acid forming an oxonium ion, reacting with nucleophiles, such as alcohol or amine, and converted into ether (or amine) derivatives.

In the absence of nucleophiles, it forms an anhydro product 4, or it can be further reduced in the presence of Et₃SiH to obtain the product 3. Compound 4 was further converted to alcohol 5a-b (5a major product) by addition of borane followed by hydrogen peroxide and aqueous NaOH (FIG. 4,

### Cytotoxicity (XTT-Assay)

All compounds were tested both towards drug-sensitive CCRF-CEM leukemia cells and their multidrug-resistant subline, CEM/ADR5000. The IC₅₀ values obtained are summarized in Table I below.

TABLE I
Cytotoxicity of artemisinin derivatives towards drug-sensitive CCRF-
CEM and multidrug-resistant CEM/ADR5000 leukemia cell lines.

| | | | Degree of |
|---|---|---|---|
| Compound | CCRF-CEM (μM) | CEM/ADR5000 (μM) | resistance |
| 1 | 148.05 ± 16.64 | 94.92 ± 30.46 | 0.64 |
| 2 | 0.87 ± 0.13 | 1.84 ± 0.31 | 2.11 |
| 3 | 240.73 ± 50.68 | 117.38 ± 8.20 | 0.49 |
| 4 | 83.36 ± 7.51 | 33.64 ± 0.68 | 0.4 |
| 5a | 156.15 ± 52.05 | 90.03 ± 4.57 | 0.58 |
| 6 | 0.55 ± 0.03 | 0.46 ± 0.03 | 0.84 |
| 7 | 0.18 ± 0.43 | 2.36 ± 0.64 | 12.83 |
| 8 | 1340.96 ± 1268.63 | 87.46 ± 96.84 | 0.06 |
| 9 | 106.00 ± 25.41 | 54.99 ± 16.00 | 0.51 |
| 10 | 12.30 ± 3.86 | 276.30 ± 213.41 | 22.46 |
| 11 | 2.68 ± 0.10 | 3.31 ± 0.24 | 1.23 |
| 12 | 6.65 ± 1.17 | 17.64 ± 4.56 | 2.65 |
| 13 | 171.00 ± 97.58 | 1333.54 ± 507.76 | 7.79 |
| 14 | 1.05 ± 0.14 | 20.75 ± 8.05 | 19.76 |
| 15 | 16.68 ± 7.34 | 7.96 ± 3.76 | 0.47 |
| 16 | 0.86 ± 0.19 | 3.34 ± 0.64 | 3.88 |

Acetal type C-10 derivatives were more active than non-acetal derivatives 3 and 4. The degree of cross-resistance of CEM/ADR5000 cells towards the various compounds ranged from 0.06 (compound 4) to 22.46 (compound 8). Substitution played an important role in C-10 derivatives. In general, alkyl side chains showed high efficacy in terms of activity and cross-resistance when compared to aromatic side chain 13 and dichloroacetate side chain 8.

Branched side chain substances possessed more activity than their straight-chain counterparts as in the case of compounds 9 and 10. When C-10 ether 15 is compared with ester 10, the activity remains the same in both cases, but ether shows slightly less drug-resistance than ester.

### Calcein Assays

As a next step, it was analyzed whether transport of calcein was affected by artemisinin and its derivatives to asses whether artemisinin-like compounds act as P-glycoprotein inhibitors.

As is shown in FIG. 5, the calcein fluorescence in CCRF-CEM and CEM/ADR5000 cell is low and not different in both cell lines after exposure to artemisinin or artesunate. This indicates that these two drugs do not act as P-gp inhibitors. In contrast, all other compounds tested led to an intracellular accumulation of calcein in multidrug-resistant CEM/ADR5000 cells, indicating an inhibition of the efflux activity of P-gp.

The EC50 values were in a range from 17.35±1.3 μM (11) to 61.8±9.62 μM (15). Intracellular calcein fluorescence increased from 916% (7) up to 3343% (14) compared to untreated controls, suggesting high affinities of these compounds to P-gp, Table II below. Well-known P-gp inhibitors were chosen as controls, e.g. verapamil and PSC-833.

TABLE II
EC50 and EC max values of artemisinin derivatives
in the calcein-AM assay using multidrug-resistant
CEM/ADR5000 cells and flow cytometry.

| Compound | EC50 (μM) | EC max (%) |
|---|---|---|
| 1 | n.d. | 114.1 ± 2.85 |
| 2 | n.d. | 114.3 ± 10.19 |
| 7 | 50.23 ± 48.5 | 916 ± 829.9 |
| 8 | 19.47 ± 5.25 | 1380 ± 199.3 |
| 9 | 36.37 ± 13.86 | 1387 ± 393.8 |
| 10 | 26.45 ± 3.12 | 1240 ± 78.2 |
| 11 | 17.35 ± 1.3 | 2224 ± 94.7 |
| 12 | 35.0 ± 8.65 | 1776 ± 292.5 |
| 13 | 17.97 ± 5.51 | 2645 ± 423.6 |
| 14 | 27.51 ± 2.59 | 3343 ± 197 |
| 15 | 61.8 ± 9.62 | 1180 ± 178.7 |
| 16 | 27.17 ± 4.69 | 1011 ± 106.3 |

n.d., not detectable

### Inhibition of Blood Brain Barrier Function

The inhibitory potential of artemisinin derivatives towards P-gp expressed in porcine capillaries was analyzed by confocal microscopy. Exposure to both compounds 8 and 15 resulted in an almost empty lumen, indicating that the P-gp substrate NBD-CSA accumulated in the endothelial cells, indicative of inhibition of P-gp. Luminal P-gp was inhibited by a well-known selective P-gylcoprotein inhibitor, PSC-833. The inhibition of luminal P-gp in porcine brain capillaries by 7 artemisinin derivatives was quantified by fluorospectrometry as shown in FIG. 6.

### Inhibition of Angiogenesis in Vivo

Eight compounds (4, 7, 8, 9, 10, 11, 12 and 15) were compared to artesunate for their anti-angiogenic potential using an in vivo zebrafish embryo model system (Table III blow). DMSO at concentrations of 0.5, 1, and 2% was used as vehicle control. No effects were observed on overall morphology, heart rate, blood flow, or angiogenesis in control embryos. The anti-angiogenic agent SU5416 was used as a positive control.

At a concentration of 10 μg/ml, SU5416 completely blocked formation of intersomitic vessels (ISVs) at 28 hpf. At 48 hpf ISVs sprouted only minimally as compared to control embryos. The heart rate was not affected by SU5416, and edema was rarely observed.

TABLE III
Anti-angiogenic effects in the zebrafish in vivo assay.

| | Conc | | | Vasc. | |
|---|---|---|---|---|---|
| Compound | [μg/ml] | N | Dead | Defects | Other |
| Artesunate | 25 | 20 | 1 | 0 | B |
| | 50 | 20 | 1 | 2 | B, E |
| | 100 | 20 | 1 | 6 | B, E |
| | 200 | 10 | 1 | 9 | B, E |
| 4 | 50 | 10 | 2 | 0 | B |
| | 75 | 10 | 5 | 0 | B, E |
| 7 | 0.1 | 10 | 1 | 1 | — |
| | 1.0 | 20 | 2 | 3 | B, E |
| | 10 | 20 | 3 | 4 | B, E |
| | 25 | 10 | 0 | 1 | B, E |
| | 50 | 20 | 4 | 8 | B, E |
| | 75 | 10 | 3 | 7 | B, E |
| | 100 | 20 | 4 | 16 | B, E |
| 8 | 1.0 | 10 | 0 | 2 | — |
| | 10 | 10 | 0 | 1 | B |
| | 25 | 10 | 0 | 10 | B, E |
| | 50 | 10 | 0 | 10 | B, E |
| 9 | 1.0 | 20 | 2 | 4 | B |
| | 5.0 | 10 | 2 | 2 | B, E |
| | 10 | 20 | 3 | 6 | B, E |
| | 15 | 10 | 1 | 9 | B, E |
| | 25 | 30 | 17 | 13 | B, E |
| | 50 | 10 | 5 | 5 | B, E |
| 10 | 0.5 | 10 | 2 | 3 | B, E |
| | 1.0 | 20 | 5 | 7 | B, E |
| | 10 | 20 | 5 | 10 | B, E |
| 11 | 1.0 | 30 | 3 | 9 | B, E |
| | 5.0 | 20 | 9 | 11 | B, E |
| | 10 | 30 | 22 | 8 | B, E |
| 12 | 1.0 | 10 | 0 | 0 | B, E |
| | 10 | 10 | 1 | 0 | B, E |
| | 25 | 10 | 9 | 0 | B, E |
| 15 | 1.0 | 10 | 0 | 0 | — |
| | 10 | 10 | 1 | 0 | B |
| | 25 | 10 | 0 | 0 | B, E |

N = total number of embryos tested (in multiples of 10);
Dead = number of dead embryos up to 48 hpf;
Vasc. Defects = number of surviving embryos with vascular defects;
Other = other defects observed: bradycardia (B) or edema (E).

The compounds tested, 7, 8, 9, 10, 11 and artesunate exhibited dose-dependent anti-angiogenic effects. Although there was some inter-experimental variability in the dose-response, compounds 7 and 8 consistently had distinct anti-angiogenic properties. Similarly, compounds 9, 10 and 11 also suppressed angiogenesis, but there was more toxicity than with compounds 7 and 8 at higher doses. Compound 12 was the most toxic at comparable doses, and a specific anti-angiogenic effect was not observed.

All of the compounds that did suppress angiogenesis were more effective, on a dose-basis, than artesunate. Of note, all compounds induced bradycardia in a dose-dependent manner, and this occurred irrespective of effects on angiogenesis. Edema, a typical consequence of heart insufficiency, coincided with the bradycardia. Preliminary experiments on rabbit hearts indicate that the bradycardia is unique to the zebrafish and not observed in mammalian models.

### Inhibition of VEGF-Induced HUVEC Proliferation

To further evaluate the anti-angiogenic potential of the present novel compounds, proliferation and survival of human umbilical vein endothelial cells (HUVECs) treated with VEGF and two compounds that were very active in the zebrafish model were assayed.

Artemisinin and VEGF alone served as control and reference compound (FIG. 7). In this assay, despite the presence of the proliferation-inducing VEGF, compounds 7 and 10 inhibited the proliferation and survival of HUVECs significantly stronger than artemisinin. Notably, the survival rate of HUVECs was very poor after more than 48 hours exposure to the compounds when VEGF was omitted.

### Discussion

By synthesizing several artemisinin-like derivatives, a range of unique compounds has been identified. It is well known that C-10 derivatives of DHA can act as pro-drugs, and that the introduction of bulky substitutes at this position decreases the rate of hydrolysis beginning with the propionate and isopropionate and different substitutes.

Thus the resultant compound derivatives may be released more slowly, potentially increasing the circulating half-life and possibly the therapeutic efficacy. Indeed, compound 10 is branch-substituted, likely reducing the rate of hydrolysis at C-10, which may contribute to its greater cytotoxicity as compared with compound 9.

Additional factors that likely impact on the activity of these compounds are solubility and conversion to DHA. In contrast to artemisinin, artesunate is water soluble and metabolized to DHA. These distinct properties may at least in part explain the greater cytotoxicity of artesunate as compared to that of artemisinin. This is exemplified the observation that C10-derivatives, which are metabolized to DHA, were more cytotoxic towards cancer cells than C9-derivatives, which cannot be metabolized to DHA. Overall, most of the new derivatives presented are not only generally more active than artemisinin, but were easily synthesized and are stable at room temperature.

In the treatment of cancer, drug resistance remains a major impediment to success. One well-characterized pathway that promotes drug resistance is the P-gp transfer system. Its relevance in clinical oncology is well known. For example, P-gp is expressed at the blood brain barrier, thereby hindering the delivery of functionally active anti-tumor drugs to the central nervous system.

Overcoming drug resistance by using compounds, such as verapamil or PSC-833, that interfere with P-gp function, have not successfully entered the clinic due to excess toxicity. Notably, artemisinin and artesunate are well-tolerated in clinical malaria studies, and it is shown herein that the present artemisinin-like compounds also modulate P-gp function, as measured with the calcein assay.

Thus, in combination with classical chemotherapeutic, P-glycoproptein substrates such as vinblastine, paclitaxel, and other anti-tumor drugs, the present novel artemisinin-like derivatives may enhance tumor cell killing, with lower toxicity, less drug resistance, and improved response rates.

As the ATP-binding cassette (ABC) transporter, P-glycoprotein, is not the only drug resistance mechanism, the question arises about the cross-resistance of artemisinin-type compounds to anticancer drugs and about the relevance of other members of the ABC transporter family.

In addition to the doxorubicin-resistant P-glycoprotein over-expressing CEM/ADR5000 cell line, artemisinin and derivatives were not cross-resistant to MRP-1-overexpressing HL60 leukemia cells and BCRP-overexpressing MDA-MB-231 breast cancer cells. They do not exhibit cross-resistance in cell lines selected for vincristine or epirubicin-resistance, nor to cell lines selected for methotrexate or hydroxyurea. Furthermore, it was found that cisplatin resistant ovarian carcinoma cells were also not cross-resistant to artemisinins.

There was no relationship between expression of P-gp, MRP1, and BCRP and the sensitivity or resistance to artemisinin and 8 different artemisinin derivatives in 55 cell lines of different tumor types (leukemia, colon Ca, breast Ca, lung Ca, prostate Ca, renal ca, brain cancer, ovarian Ca).

This result has been confirmed in another cell line panel with 39 cell lines of different tumor origin and investigation using cell lines derived from Kaposi sarcoma, medularry thyroid carcinoma, and Non-Hodgkin lymphoma. All these data indicate that artemisinin-type compounds may be active in otherwise drug-resistant cancer cells.

In the present application, it was shown that some artemisinin derivatives exert collateral sensitivity, i.e., doxorubicin-resistant P-glycoprotein over-expressing CEM/ADR5000 cells were more sensitive to these compounds than the parental wild-type CCRF-CEM cells.

Collateral sensitivity is a well-known phenomenon in multidrug-resistance cancer cells for more than three decades and led to the development of treatment strategies with compounds that selectively kill multi-drug resistant cancer cells, although the mechanisms are still poorly understood.

It has been proposed that compounds extruded by P-glycoprotein consume ATP and repletion of ATP from ADP by oxidative phosphorylation generates reactive oxygen species (ROS). ROS production may lead to increased cell killing. This view is conceivable with the fact that cell with high P-glycoprotein expression exhibit higher collateral sensitivity than cells with low P-glycoprotein levels. Artemisinin derivatives produce ROS leading to apoptosis. Hence, it can be derived that at least some of our derivatives produced more ROS than others leading to higher degrees of collateral sensitivity.

While in vitro evidence supports the notion that several of the present artemisinin-like compounds have benefits, it was important to examine their role in an in vivo model.

The Zebra fish model used supports anti-angiogenic properties of the present compounds. For example, compounds 9 and 11 suppressed intersomitic vessel (ISV) formation at concentrations as low as 1 μg/ml, above which toxicity became evident. Similarly, compounds 7 and 8 also exhibited anti-angiogenic effects, with somewhat lesser toxicity. When tested in a HUVEC proliferation/survival assay, compounds 7 and 10 were more effective at inhibiting cellular proliferation than artemisinin, despite the presence of the strong proliferation inducing growth factor VEGF.

The results of the present panel of novel artemisinine derivatives are in accord with previous reports that artemisinin, dihydroartemisinin, and artesunate act in an anti-angiogenic manner by interfering with angiogenesis-tegulating genes such as VEGFR, thrombopplastin, thrombospondin 1, plasminogen activator, matrix metalloproteinase 9 etc.

Summarizing, the present results show that the synthesized artemisinin-like compounds described are not only endowed with different properties in terms of stability and P-gp modulating activity, but that they retain potent in vivo biologic anti-angiogenic properties.

### EXAMPLE 2

A solid dosage of DHA-propionate for oral administration was prepared by direct compression or capsule filling.

DHA-propionate was recalibrated trough a 710 mm sieve for the preparation of a homogeneous mixture suitable for compression/capsule filling. The obtained particle population under 710 mm is used for further processing. A dry powder mixture was developed for direct compression aiming a 100 mg dosage (DHA-propionate) containing:
60% DHA-propionate (sieved)
70% filler mixture (Prosolv®SMCC 90)

Upon direct compression at 4 KN with an 8 mm concave punch with fraction bar, the obtained tablets (166 mg) were evaluated. These tablets have friability less than 1% (±0.12%), suitable hardness and a disintegration time of 30 seconds.

Further formulations were developed with higher ratios of DHA-propionate (80%) having also good compressibility and disintegration characteristics nevertheless with higher levels of friability. These produced tablets present a half-white coloration.

1. Compound comprising an artemisinin derivative according to the general formula: 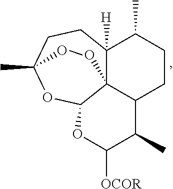
wherein R is a linear or branched C₁ to C₆ alkyl.

2. Compound according to claim 1, wherein R is selected from the group consisting of a linear or branched methyl, ethyl, propyl, and butyl.

3. Compound according to claim 1, wherein R is selected from the group consisting of CH₃, CHCl₂, C₂H₅, C₃H₇, and CH(CH₃)₂.

4. Oral formulation comprising the compound according to claim 1 and a filler.

5. Oral formulation according to claim 4, wherein the compound comprises 50% to 90% (w/w) of the formulation.

6. Oral formulation according to claim 4, wherein the formulation is a tablet or capsule form.

7. Oral formulation according to claim 6, wherein said tablet or capsule comprises 80 mg to 220 mg DHA-propionate.

8-10. (canceled)

11. Compound according to claim 1, wherein the alkyl is substituted by one or more halogens.

12. A method of treating cancer comprising administering a therapeutically effective amount of the compound according to claim 1 to a patient in need of treatment.

13. The method according to claim 12, wherein administration of the compound inhibits angiogenesis.

14. The method according to claim 12, wherein the compound is administered orally.
